(19) United States
(12) Patent Application Publication
(10) Pub. No.: US 2022/0305165 A1
(43) Pub. Date: Sep. 29, 2022

(54) Cleaning Information for Medical Device Surface Cleaning

(71) Applicant: Siemens Healthcare GmbH, Erlangen (DE)

(72) Inventors: Martin Seifert, Bayreuth (DE); Thomas Beck, Dormitz (DE); Philipp Höcht, Bavaria (DE); Tim Richter, Erlangen (DE); Sushant Mainali, Didcot (GB); Carina Moratz, Altdorf (DE)

(21) Appl. No.: 17/704,782

(22) Filed: Mar. 25, 2022

(30) Foreign Application Priority Data
Mar. 25, 2021 (DE) 10 2021 202 952.3

(51) Int. Cl. A61L 2/28; G16H 40/40; A61B 6/00

(52) U.S. Cl. CPC A61L 2/28; G16H 40/40; A61B 6/4423; A61L 2202/14; A61L 2202/24

(57) ABSTRACT

A method for providing cleaning information for at least one surface of a medical imaging device. The method includes: recording contact data indicating contact between a person and at least one surface of the medical imaging device, wherein the contact data is recorded by at least one surface sensor unit; evaluating the contact data by an evaluation unit, wherein the evaluation of the contact data comprises identifying those surfaces of the medical imaging device which indicate a contact with and/or a physical proximity to a person; and generating cleaning information for the identified surfaces on the basis of the evaluated contact data and providing the cleaning information for the surfaces.

### FIELD OF THE DISCLOSURE

The present disclosure relates to a method for providing cleaning information specific to at least one surface of a medical imaging device. In addition, the present disclosure also relates to a medical imaging device comprising a scanner unit, a patient receiving zone at least partially enclosed by the scanner unit, and a patient support and positioning device, wherein the scanner unit and the patient support and positioning device have two or more housing parts. The disclosure further relates to a computer program product comprising a computer program which is embodied to perform a method for providing cleaning information specific to at least one surface of a medical imaging device.

### BACKGROUND

During a medical imaging examination, such as, for example, a magnetic resonance examination, a computed tomography examination (CT examination), etc., and/or a preparation for a medical imaging examination, multiple instances of touching and/or contact may occur between a person, in particular the patient and/or a member of the medical staff supervising the medical imaging examination, and a surface of the medical imaging device. Following the medical imaging examination, these surfaces which the patient and/or the member of the medical operating staff have come into contact with and/or touched must be cleaned before a new medical imaging examination can be performed on another patient.

Since it is not always clearly apparent to a user, in particular to a cleaning operative, which of the surfaces of the medical imaging device was exposed to a contact with the patient and/or a member of the medical operating staff, there exists the risk that individual surfaces are not included in a subsequent cleaning activity or else that the entire device is cleaned every time, irrespective of whether cleaning is needed. To that extent, the cleaning of a medical imaging device can be particularly time-intensive. Furthermore, it is also not known to date whether a user, in particular a cleaning operative, receives direct feedback after cleaning a surface.

### SUMMARY

An object underlying the present disclosure is in particular to rectify the above-cited drawbacks when cleaning a medical imaging device. An object is achieved by means of the features of the independent claims. Advantageous aspects are described in the dependent claims.

The disclosure relates to a method for providing cleaning information for at least one surface of a medical imaging device, comprising the following method steps:

recording contact data indicating contact between a person and at least one surface of the medical imaging device, wherein the contact data is recorded by means of at least one surface sensor unit,

evaluating the contact data by means of an evaluation unit, wherein the evaluation of the contact data comprises identifying those surfaces of the medical imaging device which indicate a contact with and/or a physical proximity to a person, and

generating cleaning information for the identified surfaces on the basis of the evaluated contact data and providing the cleaning information for said surfaces.

The medical imaging device may in this case comprise for example a magnetic resonance device and/or a computed tomography device and/or a PET device (positron-emission tomography device) and/or other medical imaging devices that are deemed appropriate by the person skilled in the art. Accordingly, a medical imaging examination may also comprise a magnetic resonance examination and/or a computed tomography examination and/or a PET examination and/or further medical imaging examinations that are deemed appropriate by the person skilled in the art.

The medical imaging device preferably comprises a detector unit and/or a scanner unit which is embodied and/or configured to record medical image data during the medical imaging examination on the patient. If, for example, the medical imaging device is embodied as a magnetic resonance device, the scanner unit may comprise a main magnet for generating a homogeneous magnetic field, a gradient system for spatially encoding the recorded magnetic resonance data, and a radiofrequency antenna unit. The radiofrequency antenna unit may in this case comprise a radiofrequency antenna permanently arranged inside the scanner unit for the purpose of transmitting an excitation pulse. In addition, the radiofrequency antenna unit may also comprise a local radiofrequency antenna which is arranged around the region to be examined of the patient for the purpose of recording magnetic resonance signals.

In addition, the medical imaging device comprises a patient support and positioning device having a movable patient table. For a medical imaging examination, in particular during preparation of the medical imaging examination, the patient is initially positioned on the movable patient table of the patient support and positioning device. Furthermore, accessory units of the medical imaging device that are necessary for the pending medical imaging examination are attached to and/or arranged on the patient. An accessory unit of said type may comprise for example an ECG unit and/or an infusion unit and/or other units that are deemed appropriate by the person skilled in the art. The accessory unit may further comprise a support unit, such as, for example, a support cushion, to provide proper and/or more comfortable support and/or positioning of the patient on the patient table. In the case of a magnetic resonance examination, an accessory unit may also comprise a local radiofrequency antenna which is arranged and/or positioned around the region to be examined of the patient during a preparation of the medical imaging examination.

Already during the preparation of the medical imaging examination, multiple instances of contact and/or touching occur between persons, in particular the medical operating staff and/or the patient, and different surfaces of the medical imaging device. For example, a contact and/or touch may be the result of the patient touching side areas and a supporting section of the patient support and positioning device when sitting up and/or lying down on the patient support and positioning device. Support units, for example for supporting the head and/or legs etc., may also be touched by the patient. Furthermore, the medical operating staff may touch and/or come into contact with other surfaces on the patient table during the preparation of the medical imaging examination in order to attach a local radiofrequency antenna unit and/or an ECG unit. During this process, the surfaces of the accessory units are also touched both by the patient and by the medical operating staff.

Further instances of contact and/or touching between in particular the patient and at least one surface of the medical imaging device may also occur during the medical imaging examination. Instances of contact and/or touching between the patient and a surface, in particular contact with the surface of the enclosure of the patient receiving zone, may occur during the medical imaging examination. The patient may also come into contact with and/or touch side panels of the patient support and positioning device when leaving the patient table.

An instance of contact and/or touching between at least one surface and a person, in particular the patient and/or a member of the medical operating staff, is recorded in the form of contact data by means of the at least one surface sensor unit. The contact data in this case comprises the sensor data recorded by the surface sensor unit. The surface sensor unit is preferably configured and/or embodied to record any contact and/or touching between a person and the surface comprising the surface sensor units. The surface sensor unit may in this case include a pressure-sensitive array sensor and/or a capacitive touch sensor and/or an inductive touch sensor and/or other sensors that are deemed appropriate by the person skilled in the art for recording touching and/or contact between a person and the surface.

The method according to the disclosure may be performed for example during a medical imaging examination and/or after a medical imaging examination on a patient. Furthermore, the method according to the disclosure may also be performed and/or started when a person at risk is located in the vicinity of the medical imaging device.

A surface of the medical imaging device may in this case have a plurality of layers, one layer of the surfaces comprising the surface sensor unit. Preferably, only those surfaces of the medical imaging device in this case have a surface sensor unit which are arranged in a contact zone with a person, in particular a member of the medical operating staff and/or the patient. In particular, preferably the surfaces of the patient support and positioning device and/or the surface of an enclosure of the scanner unit encompassing the patient receiving zone and/or a surface of a front section of the scanner unit are equipped with a surface sensor unit in this case.

The evaluation unit, which is configured and/or embodied to evaluate the contact data recorded by means of the surface sensor unit, preferably comprises at least one computing module and/or a processor. For example, the evaluation may preferably be conducted continuously by means of the evaluation unit. Furthermore, the evaluation may also be conducted by means of the evaluation unit before the next medical imaging examination or also during a medical imaging examination or also after a medical imaging examination. Thus, the evaluation unit is embodied in particular to execute computer-readable instructions in order to conduct the evaluation of the contact data recorded by means of the surface sensor unit. In particular, the evaluation unit comprises a memory unit, computer-readable information being stored in the memory unit, the evaluation unit being embodied to load the computer-readable information from the memory unit and to execute the computer-readable information in order to conduct the evaluation of the contact data recorded by means of the surface sensor unit.

Most of the components of the evaluation unit may be embodied in the form of software components. In principle, however, some of said components may also be realized in the form of software-assisted hardware components, for example FPGAs or the like, in particular when there is a requirement for particularly fast calculations. Similarly, the required interfaces may be embodied as software interfaces, for example when it is simply a matter of importing data from other software components. They may, however, also be embodied as hardware-based interfaces which are controlled by suitable software. It is, of course, also conceivable that several of the cited components are realized in combination in the form of a single software component or software-assisted hardware component.

The surfaces determined and/or identified by the evaluation unit may in this case comprise surfaces that indicate having been in direct contact with a person, for example the patient and/or a member of the medical operating staff. Moreover, the surfaces determined and/or identified by the evaluation unit may also be in physical proximity to a person. For example, surfaces directly adjacent to a surface with which contact has been made may be determined as an identified surface by the evaluation unit.

The evaluation unit may further be configured and/or embodied to generate the cleaning information specific to the identified surfaces on the basis of the evaluated contact data. The cleaning information preferably comprises information on which of the surfaces, in particular the identified surfaces, were touched by and/or came into contact with a person and consequently should undergo cleaning before the medical imaging device is occupied by the next patient. Furthermore, the cleaning information may also include a type of cleaning required for the respective surface to be cleaned and/or also include an order in which the respective surfaces to be cleaned undergo cleaning, etc. Other cleaning information that is deemed appropriate by the person skilled in the art is also conceivable at any time.

Preferably, the providing of the cleaning information comprises visually presenting the cleaning information to a user, in particular to one of the medical operating staff and/or to a cleaning operative responsible for the medical imaging device, by means of a visualization unit. Alternatively or in addition, the providing of the cleaning information may also comprise providing the same to a cleaning unit in order to allow an at least partially automatic cleaning of the surfaces to be cleaned. Cleaning information is preferably provided following each medical imaging examination. Alternatively or in addition, the cleaning information may also be provided prior to a first medical imaging examination following a relatively long phase of inactivity of the medical imaging device, such as after a weekend, for example.

The disclosure advantageously enables all surfaces that are to be cleaned or have been identified to be recorded and provided for a cleaning operation and/or to a cleaning operative. As a result, a risk and/or a likelihood of a surface to be cleaned being overlooked during a cleaning operation can be reduced. This also allows the surfaces requiring cleaning to be cleaned in an efficient and time-saving manner since a laborious and time-consuming cleaning of surfaces with which no touching contact has been made can be dispensed with.

In an advantageous development of the inventive method it may be provided that the medical imaging device comprises two or more housing parts, each of the two or more housing parts having a surface sensor unit. By this means, a fast and efficient recording and/or determination of a surface to be cleaned can be realized in a constructively simple manner. In this case, all housing parts of the medical imaging device that are arranged in a front section of the medical imaging device and/or are encompassed by the patient support and positioning device and/or are arranged around the patient receiving zone have a surface sensor unit.

In an advantageous development of the inventive method it may be provided that the cleaning information for the identified surfaces is presented visually to a user by means of a visualization unit. In this case the visualization unit may comprise a display which is arranged on the scanner unit, in particular on a front section of the scanner unit. Alternatively or in addition, the visualization unit may also comprise a display and/or a monitor which is mounted on a ceiling stand inside an examination room in which the scanner unit of the medical imaging device is also arranged. Alternatively or in addition, the visualization unit may also comprise a projection unit of the medical imaging device which for example performs a projection onto a projection surface in order to present the cleaning information visually. For example, a colored light may in this case be projected onto all the surfaces of the medical imaging device that require cleaning.

This aspect of the disclosure has the advantage that an overview of all the surfaces of the medical imaging device that are to be cleaned can be displayed and/or visually presented to a cleaning operative and/or a member of the medical operating staff before the start of a cleaning operation.

In an advantageous development of the inventive method it may be provided that the cleaning information includes a cleaning plan for cleaning the identified surfaces. Preferably, the cleaning plan is displayed and/or presented to a user, in particular to a cleaning operative and/or one of the medical operating staff, by means of the visualization unit. The cleaning plan comprises in particular instructions on cleaning the identified surfaces. The cleaning plan and/or the instructions may in this case specify and/or recommend an order in which the surfaces requiring cleaning are to be cleaned, such as, for example, step-by-step instructions for cleaning the surfaces of the medical imaging device that are to be cleaned. Alternatively or in addition, the cleaning plan and/or the instructions may also specify and/or recommend a type of cleaning for the individual surfaces to be cleaned. Alternatively or in addition, the cleaning plan and/or the instructions may also specify and/or recommend a cleaning agent for the individual surfaces to be cleaned.

This aspect of the disclosure has the advantage that a user, in particular a cleaning operative, is advantageously assisted when cleaning the surfaces to be cleaned. This also enables the surfaces requiring cleaning to be cleaned in an efficient and time-saving manner.

In an advantageous development of the inventive method it may be provided that a cleaning operation in accordance with the cleaning plan is performed in a further method step. The cleaning is preferably carried out manually by a cleaning operative. In an alternative aspect, it may also be provided that the cleaning is performed automatically by means of a cleaning unit. In such a case, the cleaning unit may preferably be controlled by the evaluation unit.

The cleaning may in this comprise a mechanical cleaning of the surfaces to be cleaned. Furthermore, the cleaning may also comprise an optical cleaning of the surfaces to be cleaned, such as by beaming UV light onto the surfaces to be cleaned, for example. Furthermore, the cleaning may also comprise a wetting and/or fogging of the surfaces to be cleaned by means of a cleaning fluid, for example by means of a disinfecting fluid.

This aspect of the disclosure has the advantage that a beneficial cleaning can be achieved, in particular following completion of the medical imaging examination, and consequently a risk of infection for patients can be reduced and/or prevented.

In an advantageous development of the inventive method it may be provided that the cleaning of the identified surfaces is recorded. Recording that the surfaces requiring cleaning have been cleaned may in this case be recorded automatically and/or autonomously by means of the surface sensor unit. For example, a wetting of a surface to be cleaned with a cleaning fluid, such as, for example, with a disinfectant, may be recorded by means of the surface sensor unit based on a change in a conductivity of the surface. Alternatively hereto, the cleaning of the surfaces to be cleaned may also be recorded by means of an input unit, by way of a manual input confirming that a cleaning of a surface to be cleaned has been properly carried out. For example, the recording of the cleaning of the identified surfaces may in this case be embodied in such a way that a distinction can be made between a complete wetting and an incomplete wetting of a surface to be cleaned with a cleaning fluid.

This aspect of the disclosure has the advantage that the cleaning operation may advantageously be monitored and/or controlled and consequently errors in the cleaning of the surfaces to be cleaned, such as, for example, incomplete wetting of a surface to be cleaned by means of a cleaning agent, can also be directly recorded. In particular, it is also possible in this way to alert a user, in particular a cleaning operative, directly to an incorrectly completed cleaning of a surface and/or to instruct the user to repeat the cleaning operation.

In an advantageous development of the inventive method it may be provided that a cleaning progress of the cleaning of the identified surfaces is presented visually to a user by means of a visualization unit. For example, in this case the already cleaned surfaces and the surfaces still to be cleaned may be presented and/or displayed differently, in particular using different colors. Preferably, the cleaning progress is presented by means of a visualization unit arranged inside the examination room, such as, for example, by means of a display arranged on the scanner unit. As a result of the presentation and/or display of the cleaning progress, the user, in particular a cleaning operative and/or a member of the medical operating staff, can receive direct feedback concerning the cleaning activity just carried out. In particular, an automated proof and/or an automated logging confirming the completed cleaning of the surfaces to be cleaned may also be realized in this way.

In an advantageous development of the inventive method it may be provided that the cleaning information is stored together with the cleaning progress and/or examination information. The cleaning information is preferably stored together with the cleaning progress and/or the examination information in a database and/or a memory unit. The examination information may in this case comprise a region to be examined of the patient. Furthermore, the examination information may also comprise the type of medical imaging examination. In a magnetic resonance examination, for example, the examination information may comprise protocol information and/or sequence information, in particular which protocol and/or which sequences were used for the magnetic resonance examination. This aspect of the disclosure has the advantage that the cleaning operation may be statistically recorded and consequently an outlay on cleaning resources for cleaning the medical imaging device may also be optimized. Furthermore, this information may also be used to optimize future products, in particular medical imaging devices, in terms of cleaning overhead.

The disclosure further relates to a medical imaging device comprising a scanner unit, a patient receiving zone at least partially enclosed by the scanner unit, and a patient support and positioning device, the scanner unit and the patient support and positioning device comprising two or more housing parts. According to the disclosure, the two or more housing parts each comprise a surface sensor unit.

The medical imaging device may in this case comprise for example a magnetic resonance device and/or a computed tomography device and/or a PET device (positron-emission tomography device) and/or other medical imaging devices that are deemed appropriate by the person skilled in the art.

The surface sensor unit is in this case embodied to record a touch and/or a contact between a person, in particular a user or a patient, and the surface comprising the surface sensor unit.

In this way it is particularly easily and quickly possible to provide a cleaning operative and/or a member of the medical operating staff with an overview of which of the surfaces of the medical imaging device a person, in particular the patient and/or one of the medical operating staff, had come into contact with during the medical imaging examination and consequently also to estimate a scale of a cleaning operation, in particular a cleaning overhead. Furthermore, a cleaning operation can be optimized on the basis of information recorded in this way and consequently a particularly time-saving cleaning operation can be made possible following a medical imaging examination.

In an advantageous development of the inventive medical imaging device it may be provided that the medical imaging device comprises an evaluation unit, the medical imaging device being embodied to perform a method for providing cleaning information specific to at least one surface of a medical imaging device.

In this way all surfaces to be cleaned or all the identified surfaces may advantageously be recorded and provided for cleaning and/or for a cleaning operative. As a result, a risk and/or a likelihood that a surface requiring cleaning will be overlooked during a cleaning operation can be reduced. This also allows the surfaces requiring cleaning to be cleaned in an efficient and time-saving manner since a laborious and time-consuming cleaning of surfaces with which no touching contact has been made can be dispensed with.

The advantages of the inventive medical imaging device substantially correspond to the advantages of the inventive method for providing cleaning information specific to at least one surface of a medical imaging device, which advantages are presented in detail above. Features, advantages or alternative aspects mentioned in this regard may equally be applied also to the other claimed subject matters, and vice versa.

In an advantageous development of the inventive medical imaging device it may be provided that the medical imaging device comprises an accessory unit having a surface sensor unit, the accessory unit being connected to the evaluation unit for data exchange purposes. Preferably, the surface sensor unit is arranged and/or integrated within a surface of the accessory unit. In particular, the accessory unit is connected to the evaluation unit for the purpose of exchanging data recorded by the surface sensor unit. The accessory unit preferably comprises a unit which, for the purposes of the medical imaging examination, is arranged on the patient and/or around the patient. The accessory unit may for example comprise an ECG unit and/or an infusion unit. In addition, the accessory unit may comprise a support unit, such as a support cushion, for example, to provide proper and/or more comfortable support and/or positioning of the patient. In the case of a magnetic resonance examination, an accessory unit may also comprise a local radiofrequency antenna which is arranged and/or positioned around the region to be examined of the patient during a preparation of the medical imaging examination. Furthermore, other accessory units that are deemed appropriate by the person skilled in the art are also conceivable at any time.

This aspect of the disclosure has the advantage that information, in particular contact information and/or touch information, is also automatically recorded and/or determined for the accessory unit during the medical imaging examination. This enables the accessory unit to be taken into account also in a subsequently produced cleaning plan and prepared hygienically for a further medical imaging examination. In addition, it is also particularly advantageously possible in this way to reduce and/or prevent a likelihood of the accessory unit inadvertently not being included during a cleaning of the medical imaging device.

In an advantageous development of the inventive medical imaging device it may be provided that the surface sensor unit includes at least one pressure-sensitive array sensor. Alternatively or in addition, the surface sensor unit may also include at least one capacitive touch sensor and/or inductive touch sensor. The pressure-sensitive array sensor and/or the capacitive touch sensor and/or the inductive touch sensor may furthermore be embodied to record an area of the surface with which a person has come into contact and/or touched. In this way a particularly simple type of recording of a contact between a surface and the patient and/or the user may be recorded.

In an advantageous development of the inventive medical imaging device it may be provided that the scanner unit and/or the patient support and positioning device and/or an accessory unit comprises a surface, and a surface layer of the surface comprises the surface sensor unit. This enables a particularly space-saving arrangement of the surface sensor unit to be realized.

The disclosure further relates to a computer program product which comprises a program and can be loaded directly into a memory of a programmable computing unit and which has program means for performing a method for providing cleaning information for at least one surface of a medical imaging device when the program is executed in the computing unit. In this case the computer program may possibly require program means, e.g. libraries and help functions, in order to realize the corresponding aspect variants of the method. The computer program may in this case comprise a piece of software having a source code which still requires to be compiled and linked or which only needs to be interpreted, or an executable software code which only needs to be loaded into a suitable computing unit in order to execute.

The computer program product according to the disclosure can be loaded directly into a memory of a programmable computing unit and has program code means for performing a method according to the disclosure when the computer program product is executed in the computing unit. The computer program product may be a computer program or comprise a computer program. This enables the inventive method to be performed quickly and in an identically repeatable and robust manner. The computer program product is configured in such a way that it is able to carry out the inventive method steps by means of the computing unit. The computing unit must in this case fulfill the respective requirements, such as, for example, having a suitable random access memory, a suitable graphics card or a suitable logic unit, so that the respective method steps can be carried out efficiently. The computer program product is stored for example on a computer-readable medium or held resident on a network or server, from where it can be downloaded into the processor of a local computing unit which can be directly connected to the magnetic resonance device or embodied as part thereof. Control information of the computer program product may also be stored on an electronically readable data medium. The control information of the electronically readable data medium may be embodied in such a way that it performs an inventive method when the data medium is used in a computing unit. Thus, the computer program product may also represent the electronically readable data medium. Examples of electronically readable data media are a DVD, a magnetic tape, a hard disk drive or a USB stick on which electronically readable control information, in particular software (cf. above), is stored. When said control information (software) is read from the data medium and loaded into a controller and/or computing unit, all the inventive aspects of the above-described methods may be implemented. Thus, the disclosure may also relate to the said computer-readable medium and/or the said electronically readable data medium.

### BRIEF DESCRIPTION OF THE DRAWINGS

Further advantages, features and specific details of the disclosure will become apparent from the exemplary aspects described in the following, as well as with reference to the drawings, in which:

FIG. 1 shows an inventive medical imaging device in a schematic view,

FIG. 2 shows a cross-section through a surface of the medical imaging device, and

FIG. 3 shows a flowchart of an inventive method for providing cleaning information for at least one surface of a medical imaging device.

### DETAILED DESCRIPTION

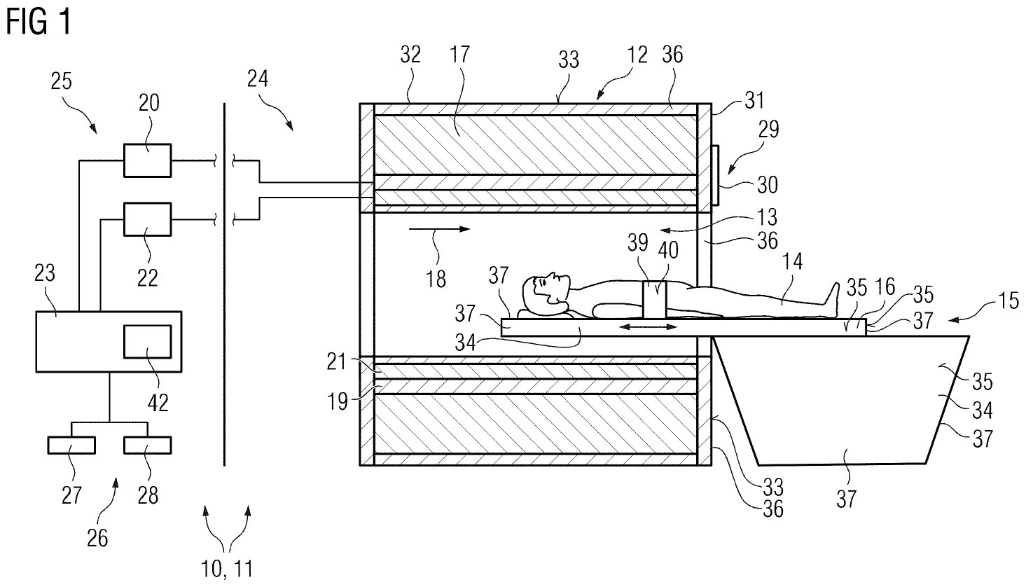

FIG. 1 shows a medical imaging device 10 in a schematic view. In the present exemplary aspect, the medical imaging device 10 is formed by a magnetic resonance device 11, the present disclosure being explained by way of example with reference to the magnetic resonance device 11. However, the present disclosure is not limited to a magnetic resonance device 11 as an aspect of the medical imaging device 10, and other aspects of the medical imaging device 10 are conceivable at any time, such as, for example, an aspect in the form of a computed tomography device and/or a PET device, etc.

The magnetic resonance device 11 comprises a scanner unit 12 formed by a magnet unit. In addition, the magnetic resonance device 11 has a patient receiving zone 13 for accommodating a patient 14. In the present exemplary aspect, the patient receiving zone 13 is embodied in the shape of a cylinder and is cylindrically enclosed by the scanner unit 12, in particular by the magnet unit, in a circumferential direction. In principle, however, a different aspect of the patient receiving zone 13 is conceivable at any time. The patient 14 can be introduced and/or driven into the patient receiving zone 13 by means of a patient support and positioning device 15 of the magnetic resonance device 11. For this purpose, the patient support and positioning device 15 has a patient table 16 embodied so as to be movable inside the patient receiving zone 13. In particular, the patient table 16 is in this case mounted as movable in the direction of a longitudinal extension of the patient receiving zone 13 and/or in the z-direction.

The scanner unit 12, in particular the magnet unit, of the magnetic resonance device 11 comprises a superconducting main magnet 17 for generating a strong and in particular constant main magnetic field 18. In addition, the scanner unit 12, in particular the magnet unit, of the magnetic resonance device 11 has a gradient coil unit 19 for generating magnetic field gradients which are used for spatial encoding during an imaging session. The gradient coil unit 19 is controlled by means of a gradient control unit 20 of the magnetic resonance device 11. The scanner unit 12, in particular the magnet unit, of the magnetic resonance device 11 additionally comprises a radiofrequency antenna unit 21 for exciting a polarization which becomes established in the main magnetic field 18 generated by the main magnet 17. The radiofrequency antenna unit 21 is controlled by a radiofrequency antenna control unit 22 of the magnetic resonance device 11 and beams radiofrequency magnetic resonance sequences into the patient receiving zone 13 of the magnetic resonance device 11.

The magnetic resonance device 11 has a system control unit 23 for controlling the main magnet 17, the gradient control unit 20 and the radiofrequency antenna control unit 22. The system control unit 23 is responsible for the centralized control of the magnetic resonance device 11, such as for performing a predetermined imaging gradient echo sequence, for example. In addition, the system control unit 23 comprises an evaluation unit (not shown in further detail) for evaluating medical image data recorded during a magnetic resonance examination.

The scanner unit 12 of the magnetic resonance device 11 is arranged together with the patient support and positioning device 15 inside an examination room 24. The system control unit 23, on the other hand, is arranged inside a control room 25. The control room 25 is embodied separately from the examination room 24. In particular, the examination room 24 is shielded from the control room 25 in terms of radiofrequency radiation. During a magnetic resonance examination, the patient 14 is situated inside the examination room 24, whereas the medical operating staff are situated inside the control room 25.

In addition, the medical imaging device 10, in particular the magnetic resonance device 11, comprises a first user interface 26, which is connected to the system control unit 23 for data exchange purposes. The first user interface 26 is arranged inside the control room 25 and has an input unit 27 and a display unit and/or visualization unit 28. Control information, such as, for example, imaging parameters, as well as reconstructed magnetic resonance images may be displayed to a member of the medical operating staff by means of the display unit and/or visualization unit 28, for example on at least one monitor, of the first user interface 26. Furthermore, information and/or parameters may be input by the medical operating staff by means of the input unit 27 of the first user interface 26 during a measurement scan.

The medical imaging device 10, in particular the magnetic resonance device 11, further comprises a second user interface 29, which is arranged inside the examination room 24. The second user interface 29 is likewise connected to the system control unit 23 for data exchange purposes. The second user interface 29 likewise has a visualization unit 30. The visualization unit 30 comprises a display which, in the present exemplary aspect, is integrated in a front housing 31 of the scanner unit 12. In this case the display may be embodied as a touch display. The second user interface 29 may further comprise a separate input unit, not shown in more detail in FIG. 1, such as, for example, operator control keys and/or control knobs integrated in the front housing 31 of the scanner unit 12. In principle, the visualization unit 30 may also comprise a monitor, which is mounted on a ceiling of the examination room 24 by means of a ceiling stand, for example, and/or a projection unit.

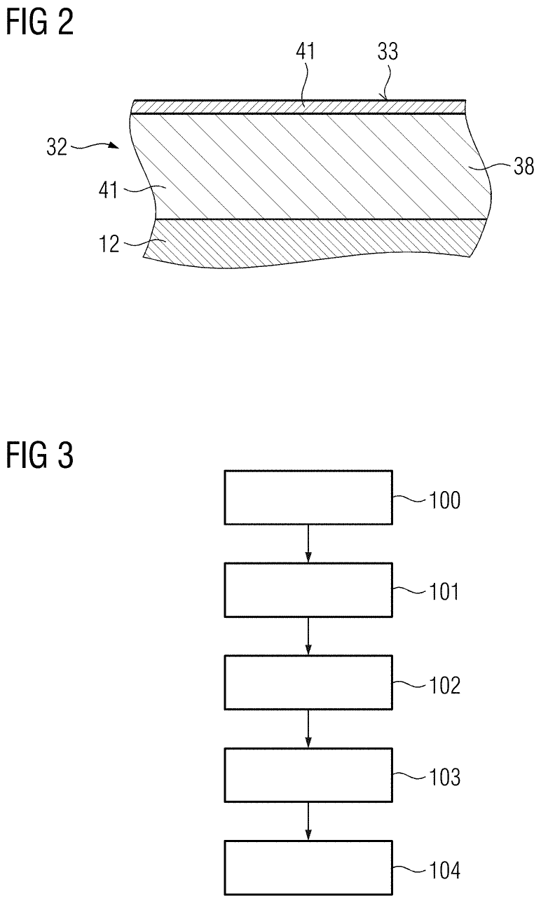

The scanner unit 12 additionally comprises a housing 32 having a surface 33. Similarly, the patient support and positioning device 15 also comprises a housing 34 having a surface 35. The housing 32 of the scanner unit 12 and/or the housing 34 of the patient support and positioning device 15 each comprise two or more housing parts 36, 37 having a surface (see FIG. 2 in this regard). The housing parts 36, 37 each comprise a surface sensor unit 38, the surface sensor units 38 in each case being embodied to record a contact and/or a touching between a person, in particular a member of the medical operating staff and/or the patient 14, and the respective surface.

In addition, the medical imaging device 10, in particular the magnetic resonance device 11, has at least one accessory unit 39. In the present exemplary aspect, the accessory unit 39 comprises a local radiofrequency coil for recording the magnetic resonance signals. The local radiofrequency coil likewise comprises a surface 40 having a surface sensor unit which is embodied to record a contact and/or a touching between the surface 40 of the local radiofrequency coil and a person, in particular one of the medical operating staff and/or the patient 14. In an alternative aspect of the accessory unit 39, the accessory unit 39 may also comprise an ECG unit and/or a support unit, such as a support cushion, for example, and/or other accessory units 39 that are deemed appropriate by the person skilled in the art.

An arrangement and/or integration of a surface sensor unit 38 inside a housing 32 is illustrated in more detail in FIG. 2. FIG. 2 shows by way of example the housing 32 of the scanner unit 12, the surface sensor unit 38 being arranged and/or integrated in an analogous manner in the housing 34 of the patient support and positioning device 15 and/or in the housing 40 of the accessory unit 39. In this case the housing 32, in particular the surface 33 of the housing 32, has a plurality of surface layers 41, one of the surface layers 41 of the surface 33 comprising the surface sensor unit 38. An outer surface layer 41 may in this case comprise a resist layer and/or a protective layer.

The individual surface sensor units 38 may contain a pressure-sensitive array sensor and/or a capacitive touch sensor and/or an inductive touch sensor and/or a proximity sensor, etc. for the purpose of recording a contact and/or a touching between a person, in particular one of the medical operating staff and/or the patient 14, and a surface.

The medical imaging device 10, in particular the magnetic resonance device 11, in FIG. 1 has an evaluation unit 42 for evaluating recorded sensor data and/or recorded contact data. The evaluation unit 42 is connected to the individual surface sensor units 38 for the purpose of a data exchange, in particular a transmission of sensor data recorded by means of the surface sensor units 38 and/or recorded contact data. In the present exemplary aspect, the evaluation unit 42 is integrated inside the system control unit 23. In an alternative aspect of the disclosure, the evaluation unit 42 may also be embodied separately from the system control unit 23.

The illustrated medical imaging device 10, in particular the magnetic resonance device 11, may of course comprise further components that are normally included in medical imaging devices 10, in particular in the magnetic resonance devices 11. Furthermore, a general principle of operation of a medical imaging device 10, in particular of a magnetic resonance device 11, is known to the person skilled in the art, so a detailed description of the further components is dispensed with.

FIG. 3 shows a flowchart of an inventive method for providing cleaning information for at least one surface 33, 35, 40 of a medical imaging device 10. The method is carried out by means of the medical imaging device 10, in particular the magnetic resonance device 11.

The method is controlled by means of the evaluation unit 42 of the medical imaging device 10, in particular the magnetic resonance device 11. For this purpose, the evaluation unit 42 has a processor and suitable control software and/or control programs. When the control software and/or control programs are executed, suitable control commands are generated in order to implement the method, such as for example, activation commands for activating the surface sensor units 38 at the start of a medical imaging examination or at the start of a preparation of the medical imaging examination.

In a first method step 100, contact data pertaining to at least one surface 33, 35, 40 of the medical imaging device 10, in particular of the magnetic resonance device 11, coming into contact with a person, in particular one of the medical operating staff and/or the patient 14, is recorded by means of the surface sensor units 38. The recording of the contact data may in this case already begin with a commencement and/or start of a preparation of the medical imaging examination, which means that contact data pertaining to contact between the medical operating staff and/or the patient 14 and the individual surfaces 33, 35, 40 is recorded already during the overall preparations for the medical imaging examination. Furthermore, the recording of the contact data may also be performed during the entire duration of the medical imaging examination, such that the recording does not terminate until the completion of the medical imaging examination, in particular until the patient support and positioning device 15 is vacated by the patient 14. Alternatively hereto, it may also be that no recording of contact data by means of the surface sensor units 38 is performed during a period in which the patient 14 is situated inside the patient receiving zone 13 of the medical imaging device 10 for a diagnostic and/or clinical imaging examination. In such a case, the individual surface sensor units 38 may also be deactivated by the evaluation unit 42 and only be reactivated as soon as the patient 14 is moved out of the patient receiving zone 13 by means of the patient table 16. The individual surface sensor units 38 independently record in each case whether a contact has occurred between a person, in particular the patient 14 and/or one of the medical operating staff, and the respective surface 33, 35, 40.

Furthermore, the surface sensor units 38 may be permanently activated in order to record a contact, thereby also enabling contacts between a person and a surface 33, 35, 40 to be recorded outside of an active phase of the medical imaging device 10. For example, a contact caused by a person entering the examination room 24 on a weekend may also be recorded in this way.

A contact and/or a touching between a person and a surface 33, 35, 40 of the medical imaging device 10, in particular of the magnetic resonance device 11, during the preparation of the medical imaging examination may be caused for example by the patient 14 touching side areas of the patient support and positioning device 15 and/or the patient table 16 when sitting up on the patient table 16 and lying down on the patient table 16. Support units, for example support cushions for supporting the head and/or the legs etc. of the patient 14 may also be touched. Furthermore, medical operating staff may touch and/or come into contact with further surfaces 35 on the patient table 16 during the preparation of the medical imaging examination for attaching an accessory unit 39, for example a local radiofrequency antenna unit and/or an ECG unit. In the process, the surfaces 40 of the accessory units 39 are also touched both by the patient 14 and by the medical operating staff. The arms of the patient 14 may also touch an enclosure of the patient receiving zone 13 and/or side areas of the patient table 16 during a positioning of the patient table 16 inside the patient receiving zone 13. Depending on the type of medical imaging examination, further instances of touching and/or contact between the patient 14 and/or the medical operating staff and a surface 33 of the medical imaging device 10 may occur.

Instances of contact and/or touching between the patient 14 and a surface 33 of the medical imaging device 10, in particular the surface 33 of the enclosure of the patient receiving zone 13, may also occur during the medical imaging examination. Following the medical imaging examination, the patient may also come into further contact with and/or touch side panels of the patient support and positioning device 15 when leaving the patient table 16.

Furthermore, further instances of contact between a person and the individual surfaces 33, 35, 40 of the medical imaging device are possible at any time. All these instances of contact, if any, are recorded by the individual surface sensor units 38 in this first method step 100.

In a subsequent, second method step 101, an evaluation of the contact data is carried out by means of the evaluation unit 42 following a completion of the medical imaging examination. For this purpose, the evaluation unit 42 has suitable evaluation software and/or evaluation programs. When the evaluation software and/or evaluation programs are executed, suitable control commands for evaluating the contact data are generated. The contact data is preferably evaluated automatically and/or autonomously by means of the evaluation unit 42. The evaluation of the recorded contact data in this case comprises identifying those surfaces 33, 35, 40 of the medical imaging device 10, in particular of the magnetic resonance device 11, which indicate a contact with and/or a physical proximity to a person, in particular the patient 14 and/or one of the medical operating staff. For example, surfaces 33, 35, 40 directly adjacent to a surface 33, 35, 40 with which contact has been made may be determined as identified surfaces 33, 35, 40 by the evaluation unit 42.

Next, in a further, third method step 102, cleaning information specific to the identified surfaces 33, 35, 40 is generated. In this case the cleaning information is generated by means of the evaluation unit 42. The cleaning information preferably comprises information as to which of the surfaces 33, 35, 40, in particular the identified surfaces 33, 35, 40, were touched by and/or came into contact with a person and/or are in proximity to a surface with which contact has been made and consequently should undergo a cleaning operation before the medical imaging device 10 is again occupied by another patient. Furthermore, the cleaning information may also include the type of cleaning for the respective surface 33, 35, 40 to be cleaned and/or also an order in which the respective surfaces 33, 35, 40 requiring cleaning are to be cleaned, etc. Particularly advantageously, the cleaning information for the identified surfaces 33, 35, 40 includes a cleaning plan for cleaning the identified surfaces 33, 35, 40.

In this third method step 102, the cleaning information generated for the identified surfaces 33, 35, 40 is then also provided. In this case the cleaning information is provided by means of the evaluation unit 42. The cleaning information provided for the identified surfaces 33, 35, 40 is in this case presented visually to a user, in particular a cleaning operative and/or one of the medical operating staff, by means of the visualization unit 30 of the second user interface 29 inside the examination room 24.

The cleaning plan comprises in particular instructions on cleaning the identified surfaces 33, 35, 40. In this case the cleaning plan and/or the instructions may specify and/or recommend an order in which the surfaces 33, 35, 40 requiring cleaning are to be cleaned, such as, for example, step-by-step instructions for cleaning the surfaces 33, 35, 40 of the medical imaging device 10 that are to be cleaned. Alternatively or in addition, the cleaning plan and/or the instructions may also specify and/or recommend a type of cleaning for the individual surfaces 33, 35, 40 to be cleaned. Alternatively or in addition, the cleaning plan and/or the instructions may also specify and/or recommend a cleaning agent for the individual surfaces 33, 35, 40 to be cleaned.

In a further, fourth method step 103, a cleaning of the identified surfaces 33, 35, 40 is then performed in accordance with the cleaning plan. The cleaning of the identified surfaces 33, 35, 40 is preferably carried out manually by a cleaning operative. Alternatively or in addition, it may also be that the cleaning of the identified surfaces 33, 35, 40 is performed at least partially automatically by means of a cleaning unit.

During the fourth method step 103, in particular during a cleaning of the identified surfaces 33, 35, 40, a cleaning of the identified surfaces 33, 35, 40 is also recorded. The cleaning of the identified surfaces 33, 35, 40 is recorded by means of the surface sensor unit 38. At the same time, a cleaning progress of the cleaning of the identified surfaces 33, 35, 40 is also presented visually. The visual presentation of the cleaning progress is realized by means of the visualization unit 30 of the second user interface 29 inside the examination room 24 for the benefit of a user, in particular a cleaning operative and/or one of the medical operating staff, so that the user receives instant feedback during a cleaning operation.

In a further, fifth method step 104, the cleaning information is stored together with the cleaning progress and/or examination information. Preferably, the cleaning information is stored together with the cleaning progress and/or the examination information in a database and/or a memory unit. The examination information may in this case comprise a region to be examined of the patient 14. Furthermore, the examination information may also comprise the type of medical imaging examination. In the case of a magnetic resonance examination, for example, the examination information may comprise protocol information and/or sequence information, in particular which protocol and/or which sequences were used for the magnetic resonance examination.

Although the disclosure has been illustrated and described in more detail on the basis of the preferred exemplary aspect, the disclosure is not limited by the disclosed examples and other variations may be derived herefrom by the person skilled in the art without leaving the scope of protection of the disclosure.

1. A method for providing cleaning information for at least one surface of a medical imaging device, comprising:
recording contact data indicating contact between a person and at least one surface of the medical imaging device, wherein the contact data is recorded by at least one surface sensor unit;
evaluating the contact data by an evaluation unit, wherein the evaluation of the contact data comprises identifying those surfaces of the medical imaging device which indicate a contact with or a physical proximity to a person; and
generating cleaning information for the identified surfaces on the basis of the evaluated contact data and providing the cleaning information for the surfaces.

2. The method of claim 1, wherein the medical imaging device has two or more housing parts, each having a surface sensor unit.

3. The method of claim 1, wherein the cleaning information for the identified surfaces is presented to a user visually by a visualization unit.

4. The method of claim 1, wherein the cleaning information comprises a cleaning plan for cleaning the identified surfaces.

5. The method of claim 4, wherein a cleaning operation in accordance with the cleaning plan is performed in a further method step.

6. The method of claim 5, wherein a cleaning of the identified surfaces is recorded.

7. The method of claim 5, wherein a cleaning progress of a cleaning of the identified surfaces is presented visually to a user by a visualization unit.

8. The method of claim 7, wherein the cleaning information is stored together with the cleaning progress or examination information.

9. A medical imaging device comprising a scanner unit, a patient receiving zone at least partially enclosed by the scanner unit, and a patient support and positioning device, wherein the scanner unit and the patient support and positioning device have two or more housing parts, and the two or more housing parts each comprise a surface sensor unit.

10. The medical imaging device of claim 9, comprising an evaluation unit, wherein the medical imaging device is configured to perform a method for providing cleaning information for at least one surface of the medical imaging device by:
recording contact data indicating contact between a person and the at least one surface of the medical imaging device, wherein the contact data is recorded the surface sensor unit;
evaluating the contact data by the evaluation unit, wherein the evaluation of the contact data comprises identifying those surfaces of the medical imaging device which indicate a contact with or a physical proximity to a person; and
generating cleaning information for the identified surfaces on the basis of the evaluated contact data and providing the cleaning information for the surfaces.

11. The medical imaging device of claim 10, comprising an accessory unit having a surface sensor unit, wherein the accessory unit is connected to the evaluation unit for data exchange purposes.

12. The medical imaging device of claim 9, wherein the surface sensor unit has at least one pressure-sensitive array sensor.

13. The medical imaging device of claim 9, wherein the surface sensor unit has at least one capacitive touch sensor or inductive touch sensor.

14. The medical imaging device of claim 9, wherein the scanner unit or the patient support and positioning device or an accessory unit comprises a surface, and a surface layer of the surface comprises the surface sensor unit.

15. The medical imaging device of claim 9, wherein the medical imaging device comprises a magnetic resonance device or a computed tomography device or a PET device.

16. A non-transitory computer program product which comprises a program loadable directly into a memory of a programmable computing unit and which has program for performing a method for providing cleaning information for at least one surface of a medical imaging device of claim 1 when the program is executed in the computing unit.
